(19) United States
(12) Patent Application Publication
(10) Pub. No.: US 2025/0269402 A1
(43) Pub. Date: Aug. 28, 2025

(54) SYSTEMS AND METHODS FOR INKING A CATHETER BALLOON

(71) Applicant: IlluminOss Medical, Inc., East Providence, RI (US)

(72) Inventors: Anthony W. O'Leary, Walpole, MA (US); Robert A. Rabiner, Dortmund (DE)

(73) Assignee: IlluminOss Medical, Inc., East Providence, RI (US)

(21) Appl. No.: 19/060,262

(22) Filed: Feb. 21, 2025

Related U.S. Application Data
(60) Provisional application No. 63/556,639, filed on Feb. 22, 2024.

(51) Int. Cl. B05D 1/00; A61M 25/10; B05C 5/02

(52) U.S. Cl. CPC B05D 1/002; B05C 5/0241; A61M 2025/1079

(57) ABSTRACT

Systems and methods are provided for applying visualization features to one or more expandable members. In some embodiments, a modular rack system is provided that is configured to receive one or more expandable members. The modular rack system comprises one or more elongate rods assemblies, with each rod assembly being sized and shaped to receive a single expandable member. An inking system is configured to apply one or more visualization features on the one or more expandable members. The one or more visualization features are applied to each of the one or more expandable members such that each of the one or more expandable members has identical visualization features thereon.

### RELATED APPLICATIONS

This application claims priority to and the benefit of U.S. Provisional Patent Application No. 63/556,639, filed Feb. 22, 2024, the contents of which is incorporated herein by reference in its entirety.

### FIELD

The embodiments disclosed herein relate to medical devices for use in repairing a weakened or fractured bone, and more particularly to internal bone fixation devices and methods of using these devices for repairing a weakened or fractured bone using visualization techniques, including radiopaque marking on the device.

### BACKGROUND

Various techniques are used to add inked features to medical devices, including contact printing, screen printing, inkjet dispensing, and appliqué techniques. These techniques can present challenges, especially when used to apply material to medical devices used inside the body due to dimensional constrains of the devices, specifically lateral dimensions that are typically constrained to the millimeter or centimeter regime.

### SUMMARY

In some embodiments, a device is provided that includes a modular rack system configured to receive one or more expandable members, The modular rack system includes one or more elongate rods assemblies, with each rod assembly being sized and shaped to receive a single expandable member. An inking system is configured to apply one or more visualization features on the one or more expandable members, wherein the inking system is configured to apply the one or more visualization features to each of the one or more expandable members such that each of the one or more expandable members has identical visualization features thereon.

In some embodiments, the one or more visualization features on each of the one or more expandable members has substantially equal line thickness and width along a length of each of the one or more expandable member.

In some embodiments, the inking system further includes an inking head assembly configured to move over the modular rack system for applying the one or more visualization features to the one or more expandable members.

In some embodiments, the device further includes a robotic arm configured to slidably move along a length of a frame positioned over the modular rack assembly to pass over the expandable members to apply the one or more visualization features.

In some embodiments, the one or more elongate rod assemblies includes: a rod configured to receive and support one of the one or more expandable members; a first end cap configured to couple the rod to a first end of the modular rack system; and a second end cap configured to couple the rod to a second end of the modular rack system.

In some embodiments, the device further includes an air manifold positioned on a first end of the modular rack assembly, the air manifold configured to couple to each rod assembly to provide air for expansion of the one or more expandable members. In some embodiments, the air manifold is configured to provide air during application of the one or more visualization features.

In some embodiments, the device further includes a drive mechanism coupled to each rod of the rod assemblies on which each of the one or more expandable members is positioned that is configured to rotate each of the one or more expandable members. In some embodiments, the drive mechanism is configured to rotate each of the one or more expandable members at an identical speed. In some embodiments, the drive mechanism includes a series of gears configured to rotate the one or more rod assemblies. In some embodiments, the drive mechanism includes a series of gears and a belt, the belt being configured to rotate the series of gears to rotate the one or more rod assemblies.

In some embodiments, a device is provided that includes a modular rack system configured to receive one or more expandable members. The modular rack system includes one or more elongate rods assemblies, with each rod assembly being sized and shaped to receive a single expandable member. An inking system is configured to apply one or more visualization features on the one or more expandable members. A drive mechanism is coupled to each of the elongate rod assemblies, and the drive mechanism is configured to rotate each elongate rod assembly uniformly such that each expandable member is rotated at an identical speed. The one or more visualization features are applied to each of the one or more expandable members such that each of the one more expandable members has identical visualization features thereon.

In some embodiments, the drive mechanism includes a series of gears configured to rotate the one or more rod assemblies. In some embodiments, the drive mechanism includes a series of gears and a belt, the belt being configured to rotate the series of gears to rotate the one or more rod assemblies. In some embodiments, the inking system further includes an inking head assembly configured to move over the modular rack system for applying the one or more visualization features to the one or more expandable members.

In some embodiments, a method is provided that includes preparing one or more expandable members for receiving one or more visualization features by applying the one or more expandable members to one or more rod assemblies, positioning the one or more rod assemblies on a modular rack, rotating the one or more expandable members using a drive mechanism coupled to the rod assemblies and the modular rack, and applying one or more visualization features to the one or more expandable members. The one or more visualization features being applied to each of the one or more expandable members simultaneously such that each of the one more expandable members has identical visualization features thereon.

In some embodiments, the method further includes inflating each of the one or more expandable members using an air manifold coupled to the rod assemblies. In some embodiments, the one or more visualization features are applied during inflation of the one or more expandable members.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 illustrates an exemplary embodiment of an inking device configured to deposit radiopaque material on one or more balloons as parts of a bone fixation device;

FIGS. 2A and 2B illustrate an exemplary embodiment of an inking device and a modular rack configured to be positioned on a base of the inking device;

FIG. 3 illustrates an exemplary embodiment of a modular rack system for holding one or more balloons for inking;

FIG. 4A illustrates an exploded view of an exemplary embodiment of one or a plurality of rod assemblies for holding a balloon in the modular rack system of FIG. 3;

FIG. 4B illustrates an assembled view of an exemplary embodiment of one or a plurality of rod assemblies for holding a balloon in the modular rack system of FIG. 3;

FIG. 4C illustrates a cross-sectional view of an exemplary embodiment of one or a plurality of rod assemblies for holding a balloon in the modular rack system of FIG. 3;

FIG. 5 illustrates an exemplary embodiment of an end cap of a rod assembly for coupling to an air manifold;

FIG. 6 illustrates an exemplary embodiment of an air manifold configured to deliver air to each balloon on the modular rack system;

FIGS. 7A and FIG. 7B illustrate an exemplary embodiment of a drive system for rotating the rod assemblies of the modular rack system;

FIGS. 8A and FIG. 8B illustrate an exemplary embodiment of a drive system for rotating the rod assemblies of the modular rack system;

FIGS. 9A and 9B illustrates an exemplary embodiment of a robotic inking head for depositing ink on one or more balloons;

FIGS. 10A and 10B illustrates exemplary embodiments of radiopacity of jetted ink;

FIGS. 11A, 11B, 11C and FIGS. 12A, 12B, and 12C illustrate examples of the use of X/Y/Z orientation of the piezo electric head to ensure line conformity of deposited ink;

FIGS. 13A and 13B illustrate an exemplary embodiment of a direct inking process;

FIG. 14 illustrates an exemplary computer system for use in connection with the systems and methods of the present disclosure;

FIG. 15 illustrates an exemplary embodiment of a preparation tank for preparing the balloons for inking;

FIG. 16 illustrates an exemplary embodiment a corona discharge mechanism;

FIG. 17 illustrates an exemplary embodiment of a method for depositing ink on a substrate;

FIG. 18 illustrates an exemplary embodiment of a bone repair catheter assembly;

FIGS. 19A, 19B, 19C, and 19D illustrates an exemplary embodiment of a bone repair sequence;

FIG. 20A illustrates an exemplary embodiment of balloon after the inking process;

FIG. 20B shows an exemplary fluoroscopic image of a using the balloon of FIG. 20A;

FIG. 21 illustrates an exemplary embodiment of a medical heater suitable for ablation procedures;

FIG. 22 illustrates an exemplary embodiment of a printed flexible electrode array; and

FIGS. 23A and 23B illustrate exemplary embodiments of a printed polymeric stent.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Systems and methods for adding visualization features or other features to medical devices, for example, for repairing a weakened or fractured bone, are disclosed herein. The devices disclosed herein act as internal bone fixation devices and include a delivery catheter terminating in a releasable conformable member, expandable member, or balloon. During a procedure for repairing a fractured bone, the balloon is placed within an inner cavity of a fractured bone in a deflated state. Once in place, the conformable member is expanded from a deflated state to an inflated state by the addition of at least one reinforcing material. The at least one reinforcing material is subsequently hardened within the conformable member using a light source. The hardened conformable member may then be released from the delivery catheter and sealed to enclose the at least one reinforcing material within the conformable member. The hardened conformable member remains within the inner cavity of the fractured bone and provides support and proper orientation of the fractured bone resulting in the repair, healing, and strengthening of the fractured bone.

In some embodiments, direct capillary printing (DCP) can be used to add the visualization features to medical devices. DCP is a fabrication methodology of directly depositing controlled amounts of a “flowable” material, referred to as “ink,” onto a surface through a small dispensing orifice according to a digitized tool path. For example, one or more radiopaque markers in the form of an ink can be placed at various locations along the balloon. The ink, using radiopaque materials such as barium sulfate, tantalum, or other materials known to increase radiopacity, allows a medical professional to view the device using various techniques, including fluoroscopy. The ink can also provide visibility during inflation of the balloon to determine the precise positioning of the balloon and the device during placement and inflation. The ink permits visualization of any voids that may be created by air that gets entrapped in the balloon. The ink can permit visualization to preclude the balloon from misengaging or not meeting the bone due to improper inflation to maintain a uniform balloon/bone interface.

In some embodiments, a robotic inking device is provided that is configured to apply radiopaque ink using a piezo-electric driven non-contact jet valve delivery system. The jet valve application can be faster and more precise in the dispensing of the radiopaque ink. The application process allows for multiple balloons to be prepared at the same time and with the same inking pattern thereon, with each inking pattern having a substantially equal line thickness and width along the length of each balloon.

The type of radiopaque ink can vary. In some embodiments, the radiopaque (RO) ink in its liquid form consists of a high concentration of tungsten particles (particle size range within 0.1 to 75 microns) mixed with a polymer binder and a solvent. In some embodiment, the polymer binder used is Carbothane™ in solvent solution. The tungsten can be mixed and then applied to the balloon. In some embodiments, the cured RO ink (solvent removed) consists of about 93% tungsten by weight or about 45% by volume.

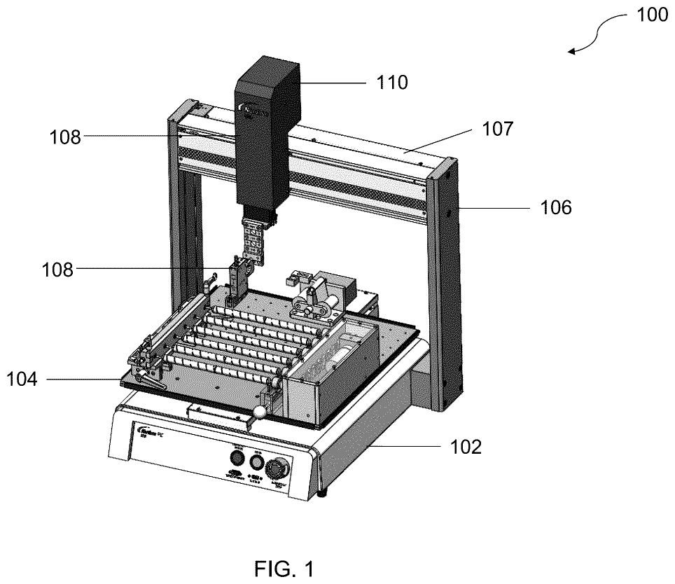

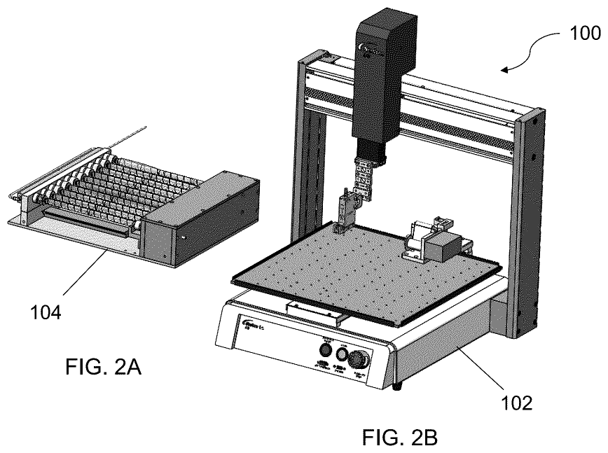

In some embodiments, an inking device 100, shown in FIG. 1 and FIGS. 2A-2B, includes a base 102 configured to receive a modular rack system 104, and a frame 106 configured to support an inking head assembly 108 that can move over the modular rack system 104 along the frame and the base for applying one or more visualization features, for example in the form of an ink, to one or more balloons. The frame 106 includes an upper portion 107 for supporting the inking head assembly 108, and a first and second side support for coupling the base and the ends of the upper portion of the frame 106. The inking head assembly 108, which comprises a robotic arm 110, is configured to slidably move along the length of the upper portion 107 of the frame 106 to pass over the balloons positioned on the modular rack assembly to apply the visualization features or other features (for example, using ink) to the one or more balloons.

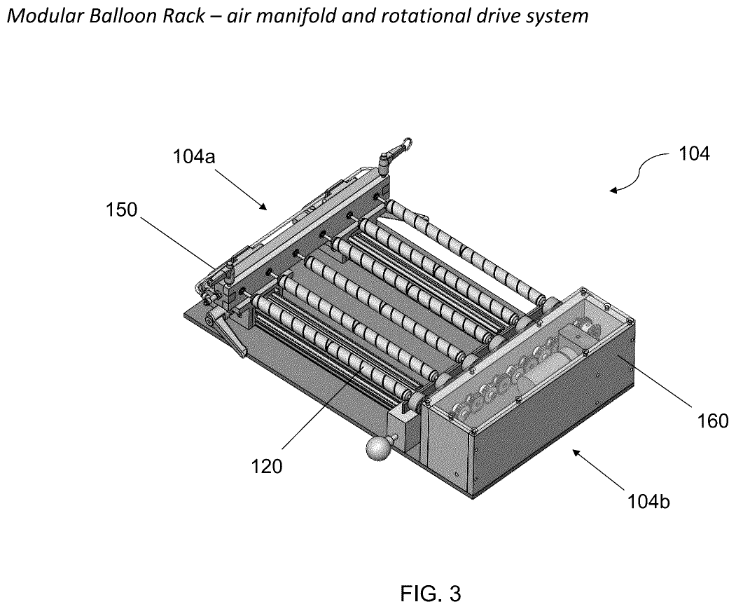

The modular rack system 104, shown in FIG. 3, is configured to hold one or more balloons such that one or more balloons can be prepared to be treated at the same time, thus increasing speed and efficiency of the ink application process. In some embodiments, the rack system 104 includes a plurality of elongate rods assemblies 120, or mandrils, that extend from a first end 104a of the rack system to a second end 104b of the rack system. Each rod assembly 120 is sized and shaped to receive a single balloon thereon and includes balloon fittings for balloon rotation and air expansion during the process of applying the visualization features on the surface of the balloons. A person skilled in the art will understand that any number of balloons can be held by the modular rack system, and that the size of the modular rack system can vary to accommodate different numbers of balloons.

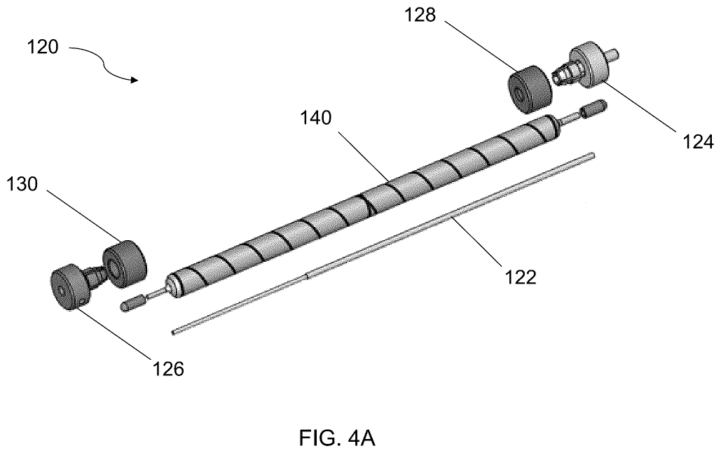

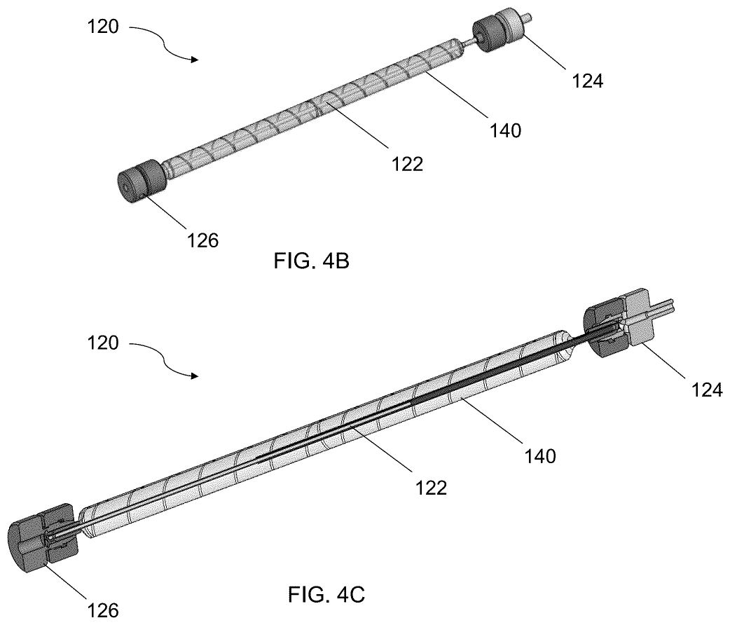

Each rod assembly is shown in more detail in FIGS. 4A, 4B, and 4C. FIG. 4A illustrates an exploded view of an exemplary embodiment of the rod assembly 120, FIG. 4B illustrates an assembled view of the rod assembly 120, and FIG. 4C illustrates a cross-sectional view of the rod assembly 120. The rod assembly 120 comprises an elongate rod 122 that is sized to hold an individual ballon 140, or expandable member, thereon. The elongate rod 122 includes a first end cap 124 and a second end cap 126 for removably or detachably coupling the elongate rod to the rack system. The first end cap and the second end cap each include a compressive fitting that is positioned on each end of the elongate rod after placement of the balloons on the rods of the rack system to lock the balloons in place during inking. In some embodiments, the first end cap 124 includes a compressive fitting 128 that is configured to couple the first end cap 124 to the first end of the rack assembly. In some embodiments, the second end cap 126 includes a compressive fitting 130 that is configured to couple the first end cap 126 to the second end of the rack assembly.

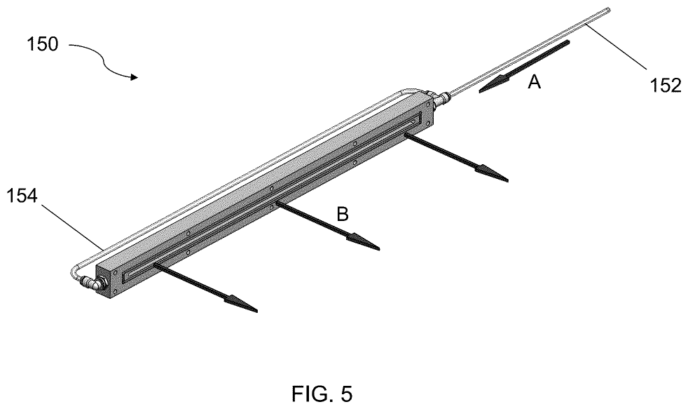

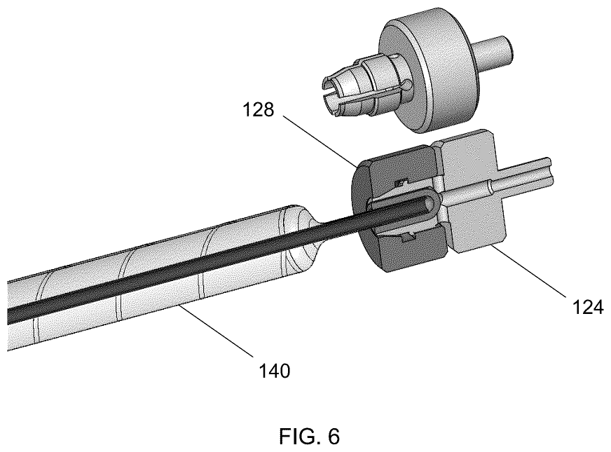

In some embodiments, the modular rack system can include features to aid in supporting the shape of the balloon to facilitate proper inking. For example, inking can be more precise and consistent when the balloon is firm when inflated. In some embodiments, the use of air inflation and/or pressure on the balloons can be used to resolve deformities or eccentricities experienced over the length of an uninflated balloon. As shown in FIG. 3 and in more detail in FIG. 5, the rack system 104 can include a mechanism, such as an air manifold 150, to pressurize the balloons positioned in the rack system. The air manifold 150 is positioned at the first end 104a of the modular rack assembly 104 and is configured to couple to the modular rack assembly to deliver air to each balloon positioned on each of the rod assemblies seated in the modular rack assembly. The air manifold 150 can include a length of tubing 152 to deliver air, such as pressurized ambient air, to the rack system 104. The air can travel through the tubing 152 in the direction of arrow A and into the air manifold such that the air can exit the air manifold in the direction of arrow B into each of the rods assemblies 120 to inflate each balloon. The first end cap 124 with the compressive fitting, as shown in FIG. 6, can include a mechanism such that, after placement of the balloons 140 on the rods, air can pass from the air manifold in the rack system through the first end cap 124 and into the balloons for inflation of the balloons during inking.

In some embodiments, the rack system provides a mechanism to rotate the balloons during the inking process. The system can include a drive mechanism 160, shown in FIG. 3 that acts as a motor and is coupled to each rod of the rod assembly on which each balloon is positioned and is configured to rotate each balloon. In some embodiments, the drive mechanism 160 is configured to rotate each balloon uniformly such that each balloon is rotated at an identical speed. When the drive speeds are the same over the entire modular rack system, it can ensure that the line, position, thickness, and/or deposition of the ink from balloon to balloon are identical.

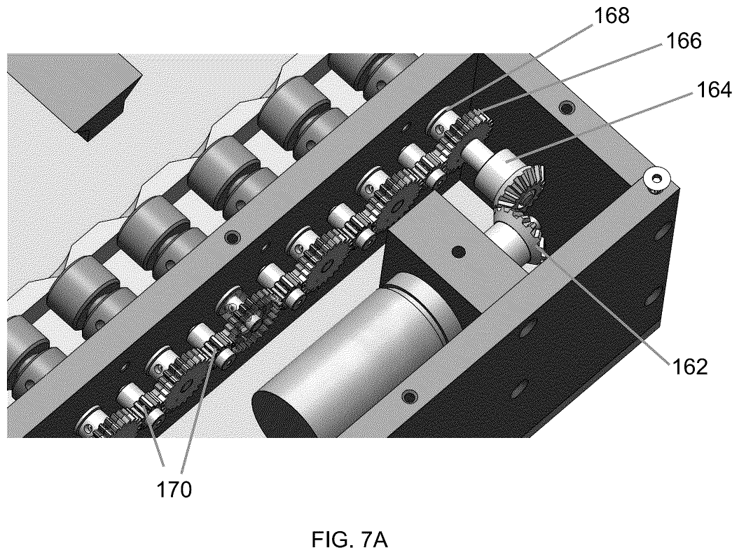

In some embodiments, the drive mechanism utilizes a series of gears, as shown in FIGS. 7A and 7B. To allow each rod assembly to rotate, the drive mechanism includes a gear associated with each rod assembly. Each rod assembly gear is configured to attached to the rear of the rotator of each rod assembly for each balloon (gears on the rear of the balloon mandrel). As shown in FIG. 7A, the drive mechanism includes a first gear 162 (or motor gear) that is configured to engage with a motor 161. The first gear 162 engages with a second gear 164 (or drive gear). The first gear and the second gear are positioned at a 90 degree angle relative to one another. The second gear of the drive mechanism is configured to interface with or engage with the first rod assembly gear 166. Each of the rod assembly gears are coupled to each corresponding rod assembly along an axel 168 such that the rotation of the first gear, the second gear, and the rod assembly gear rotates the corresponding rod assembly.

Between each of the rod assembly gears, a spacer gear 170 is positioned, which acts as a drive connector to the next gear on the balloon mandrel. The spacer gears act as a means to have all of the balloons rotating in the same direction, as connecting the two balloon drive gears directly would reverse the rotation. The connection of the gears reduces “slop” movement and/or “lag” in the drive process.

FIG. 7B illustrates an exemplary embodiment of the gear train of the drive mechanism of FIG. 7A, illustrating the direction of rotation of each component of the drive train, from the motor 161 to the first gear 162, the second gear 164, and the series of rod assembly gears and spacer gears 170. While FIG. 7B illustrates four rod assembly gears configured to rotate four balloons and 3 spacer gears, it will be understood that any number of rod assembly gears and spacer gears can be used to accommodate any number of rod assemblies and balloons.

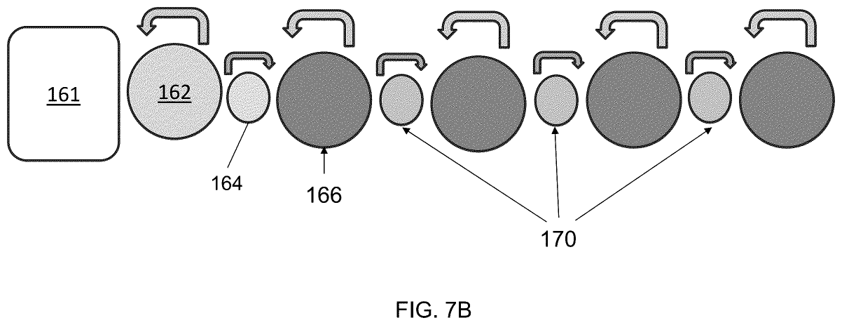

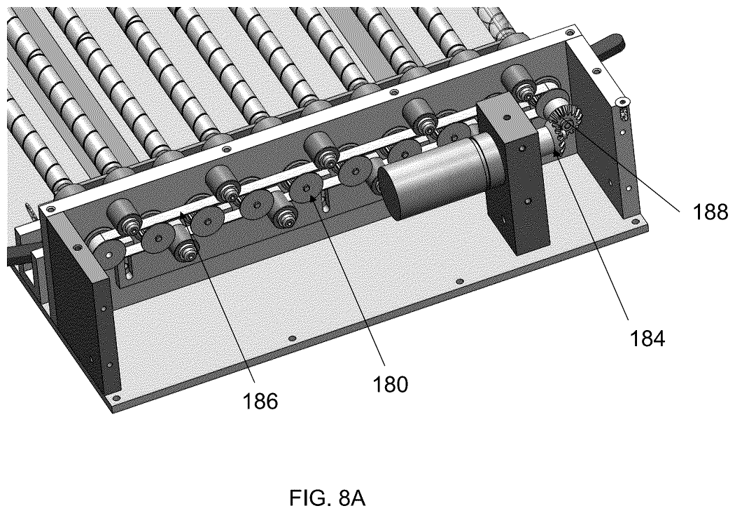

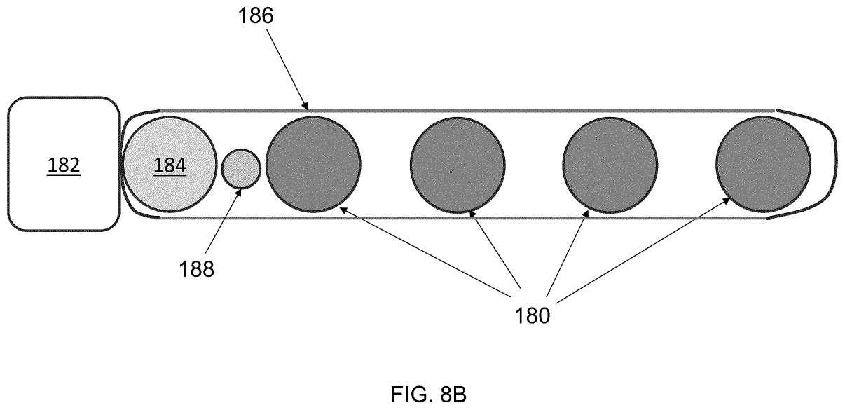

In some embodiments, the drive mechanism utilizes a belt drive, as shown in FIGS. 8A and 8B. The belt drive can be in the form of a geared belt that engages with a geared drive wheel on the motor. The belt is positioned to surround all of the rear mandrel gears. Similar to the drive mechanism shown in FIGS. 7A and 7B, the drive mechanism includes a plurality of rod assembly gears 180 corresponding to each rod assembly positioned on the modular rack. A motor 182 couples to a motor gear 184, and then to a drive gear 188 positioned to interface with motor gear at a 90 degree angle. The motor gear is configured to rotate a gear drive belt 186 that is positioned around the plurality of rod assembly gears 180. Each rod assembly gear is coupled to one of the rod assemblies, and includes features thereon for engaging with the drive belt 186. The rotation of the drive belt causes rotation of the rod assembly gears, the rod assemblies, and the corresponding balloons. This allows each balloon to rotate simultaneously and with the same speed. While FIG. 8B illustrates four rod assembly gears configured to rotate four balloons, it will be understood that any number of rod assembly gears can be used to accommodate any number of rod assemblies and balloons.

In some embodiments, the rack system can include a hinge mechanism at a first end of the rack system such the rack system can pivot to allow for placement of the balloons on each rod. In some embodiments, the air manifold is configured to attach to the first end of the modular rack system on a hinged component. In some embodiments, the hinged component is configured to rotate 90 degrees to allow the rod assemblies to point upward. This allows for the mounting of the balloons on the rods of the rod assemblies. In some embodiments, the hinged component and the air manifold are unitary. For example, they become one unit when the system is assembled. In some embodiments, the hinged component can sit on a movable track that allows the rod assemblies to be advanced from the first end to the second end, so that after the balloons are mounted on the hinged side (the first end), the rod assemblies can be advanced and inserted into the second end to couple the rod assemblies to the drive mechanism. For example, the rod assemblies can be mounted into the hinged component 154 shown in FIG. 5.

In some embodiments, the rod assemblies are able to rotate 90 degrees from horizontal to vertical. The balloons can be mounted and attached to the air system of the air manifold. The hinge can then be lowered to move the balloons into a horizontal position, and the rack can be advanced from the first end towards the second end with the rod assemblies, or mandrels, and the balloons thereon being coupled to the drive side of the system.

In some embodiments, the inking head assembly utilizes piezo-electric jetting of the ink, which allows for immediate on and off of the ink process and for complex designs to be made on the surface of the balloons. The current direct printing method utilizes pressure to drive the ink through an orifice in a tube (it will be noted that the start-up phase of the inking of each balloon has a bubble of ink that needs to be remediated). Jetting has no bubble or residual ink at the tip, and as the drive mechanism is immediate on/off, there is no lagging pressure.

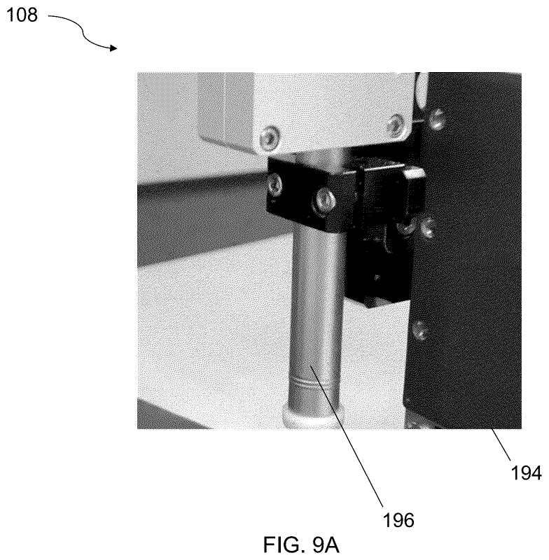

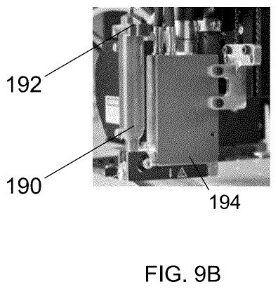

As shown in FIGS. 9A and 9B, the inking head assembly 108 can include an inker/pressurized ink cartridge, a drive system (for example, a piezo-electric drive system), and a camera.

In some embodiments, the ink cartridge 190 is formed from a plastic, glass, or other material. The ink cartridge is configured to hold the media to be applied to the balloon. The cartridge is coupled to the drive system 194 with a cap, screw, luer, or other attachment mechanism 192 that ensures that the system is engaged in a manner that does not allow the pressure to escape from the attachment mechanism between the ink cartridge and the drive system. For example, as the system is pressurized to ensure that consistent media is provided to the drive system, there should be no leakage, for both system efficiency and pressure leakage would cause the media to escape the system.

The camera 196 on the system provides two functions. First, the camera can be used to orientate the system. For example, the camera can be configured to function to determine the position of the inking head assembly to find and/or define a specific point on the rack in an X, Y, and/or Z orientation so that when the inking commences, the system is positioned at the correct position for the deposition of ink. For example, the camera can look for a registration point that has been defined in the system during set up.

Second, the camera can be used after the locational activities to function as an inspection scope. In some embodiments, the camera can provide the user with a real-time, close-up view of the ink that is being applied to the balloon. As the balloons are a component of a medical device, they can be assembled/constructed in a fashion to minimize contamination, contact, or other handling by the operator. The camera provides a means for real time inspection.

In some embodiments, the camera can be CMOS. The visualization is a lens system conventional optic train, or fused fiber. The system is designed to be small in diameter so as not to impede the system size/motion/movement of the racks.

Referring again to FIG. 1, the inking head assembly 108 can be mounted on a robotic arm 109 positioned on the frame 106. The inking head assembly is configured to move in all three dimensions such that ink can be applied to any of the balloons mounted in the modular rack assembly. For example, the robotic arm is slidably coupled to a top portion of the frame such that the robotic arm can move laterally along the frame to apply the ink along a length of each balloon positioned in the modular rack. While the figures show a single inking head attached to the robotic arm that can move to ink each balloon placed on the rack, it will be understood that the system can include more than one inking head to ink multiple balloons simultaneously or as a backup inking head. In some embodiments, multiple inking heads can be used to deposit different materials onto the surface of the balloon. For example, a first head can apply ink, and a second head can apply a secondary material. Secondary materials can include, but are not limited to, a clear coating or cover to the surface of the balloon.

In some embodiments, the secondary materials can include metal inks, ostensibly metal films, and polymers. These secondary materials can be used in the construction of a force sensing resistor, which utilizes layers of material that detect force and/or compression. For example, the metal films can be made of printed, piezoresistive silver and carbon ink. When the two layers of conductive ink press together, the electrical resistance decreases, and the signal can be communicated to a processor or reader.

For example, many medical devices require more than one type of ink in order to achieve the desired product functionality. Different types of inks can be required on a single device, such as a single-phase liquid ink or a multi-phase ink. A single-phase liquid is formulated by dissolving a solid in either a single solvent or a mixture of solvents, referred to as the solvent system. A multi-phase ink contains both a liquid phase as described above, as well as a collection of fine solid particles generically referred to as the filler. While multi-phase inks typically involve a much greater complexity of formulation, they also allow for important functional behaviors that are difficult or impossible to achieve in single-phase inks. The ink characteristics contributed by the filler materials are determined by the properties of the particles such as composition, morphology, surface species, and size distribution. As with single-phase inks, multi-phase inks are typically thermally cured or fired if the binders were solubilized and optically or chemically cured if they were not.

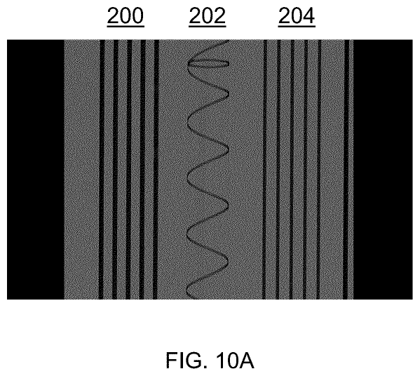

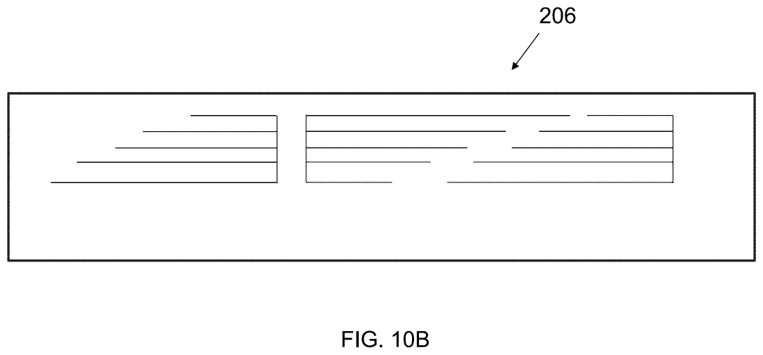

The inking head can apply the RO ink to the balloon in various patterns as the robotic arm moves the inking head along the surface of the balloon. In some embodiments, the balloons are rotated on the rods of the rack during the inking process such that the ink is delivered in a continuous, spiral design over the external balloon surface as the inking head is moved down the length of the balloon. FIGS. 10A and 10B illustrates various examples of patterns that can be inked onto the surface of the balloons, including stripes 200, 204 of different thicknesses and a spiral pattern 202. In some embodiments, a spiral, as shown in FIG. 10A, can be used as it provides a doctor, such as an orthopedic surgeon, with the best view of the implant within the body, such as in a bone canal, towards the determination of location/space/filling of the balloon.

In some embodiments, a series of stripes 206 can be applied to the balloon, as shown in FIG. 10B. For example, a force sensing balloon can include a pattern of a series of stripes that are running down the length of the balloon. The stripes can be parallel to each other and have the lengths of the stripes opposite in length in a plurality of sections along the length and/or circumference of the balloon. Thus, as forces are applied to the implant and/or balloon, measurements can be taken in small discrete areas relative to the striped pattern.

In some embodiments, the thickness of the deposition of the ink can be important. For example, thicker materials can be more radiolucent for providing potential visualization and determination of balloon position, placement, and/or orientation in the body due to the denser view on the x-ray or fluoroscopy machine.

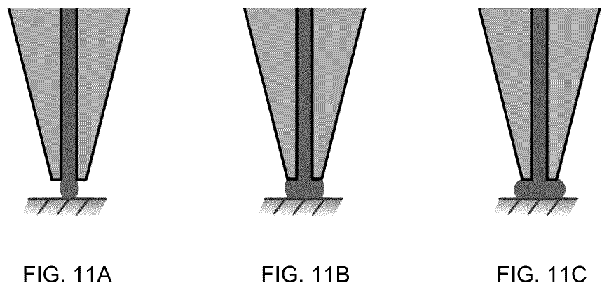

The non-contact system prevents any potential damage to the balloon. The printed ink trace should have a uniform thickness and should not slump or spread across the substrate. The pen tip is not touching the substrate and is, in fact, riding on the dispensed ink. FIGS. 11A, 11B, and 11C show exemplary embodiments of how various trace widths arise. Low net pen tip force causes trace width to favor the bore diameter of the pen tip. Carefully balanced pen tip force causes ink to flow out and be pinned by surface tension to the pen tip edges. Excessive pen tip force causes ink to squeeze out beyond the tip perimeter. In addition, inks with low viscosity or low surface tension can spread far beyond the bounds of the outside diameter of the pen tip.

There are a variety of corrective mechanisms that can be used to work around certain substrate deficiencies. Some of these can be incorporated in the printing hardware and others can be implemented through process integration or the pattern design itself. For example, as explained above, cylindrical substrates such as tube-based and catheter-based devices can be straightened by either inserting a mandrel into a central lumen or by holding the part in tension. For example, substrates generally exhibit some degree of roughness and out-of-plane behavior at the local or even microscopic level.

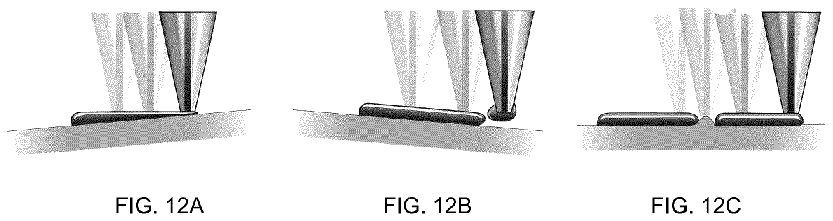

Shallow undulations can be visualized as localized regions of tilt. Different types of printing anomalies can occur depending on whether the tilt is “uphill” or “downhill,” as depicted in FIG. 12A and 12B. An upward tilt can cause the pen tip substrate separation distance to diminish from its equilibrium value if the height of the pen tip is not adjusted quickly and accurately. The result can be excessive trace width, reduced trace thickness, and transfer of ink onto the sides of the pen tip. In the most extreme case, the pen tip comes into hard contact with the surface and the ink flow is instantaneously valved off, often resulting in a void in the feature. Conversely, a downward tilt of the substrate causes the pen tip-substrate separation to increase beyond its equilibrium value.

If uncorrected, the printing behavior can transition to a mode that more resembles pure extrusion of ink from the orifice. The exact manner in which the extruded thread of ink interacts with and lays down onto the surface can be become unpredictable, resulting in changes in trace width and height, edge acuity, and even trace location. In the most extreme case, the extruded ink thread might neck down and separate. Surface tension will then cause flowing ink to adhere to the pen tip and form a droplet and in the meanwhile printing onto the substrate ceases.

There are several hardware strategies to deal with tilt in the substrate. The substrate could be carefully scanned, and a three-dimensional map stored and used to generate pen tip height signals during the subsequent printing process. Surface scanning can be performed optically or mechanically. With the ink flow turned off, the pen tip itself can be used as a mechanical stylus. The downside to this approach is the time required to perform the scan, which might require microscopic resolution depending upon the application. Alternatively, a means to actively ensure constant height of the pen tip can be employed. This could be accomplished by taking continuous localized height measurements or by analyzing forces at the pen tip and employing a feedback loop to stabilize them.

High frequency, high magnitude variations in the substrate surface such as asperities and pits can cause similar issues. FIG. 12C shows the case of a surface asperity severe enough to cause a momentary cessation of ink flow. If the asperity were due to solid particle contamination there is the additional possibility that the particle could be dragged by the pen tip and transported a considerable distance, creating printing anomalies along the way.

A number of methods have been used to transport the ink from its reservoir onto the substrate. When the reservoir takes the form of a conventional syringe, the simplest way to generate force on the ink is to seal the top of the syringe and apply a controlled pressure. These so-called “air over” systems typically use compressed air or nitrogen and can be relatively inexpensive to set up and operate. They are extensively used to dispense adhesives and other low to moderate viscosity materials. In some embodiments, a pump in the form of an auger or Archimedes screw is used to drive the ink out of a fine nozzle. Ink flow is determined by the rotational speed of the screw, which can be precisely controlled. In addition, auger-based systems have an inherent ability to rapidly start and stop the ink flow because stored energy and hysteretic behavior in the print head are minimized. In some embodiments, a pump in the form of a positive displacement piston-and-cylinder arrangement can be used. In its simplest embodiment the single piston-and-cylinder acts as both the reservoir and the pump. In some embodiments, an enhancement of the piston-and-cylinder pump is the use of a dual piston mechanism. In this configuration, there is a dedicated reservoir which functions independently of the cylinders. The cylinders alternatively draw in ink from the reservoir and then dispense it to the pen tip. A multi-port valve is synchronized to the motion of the coupled pistons. This dual piston-and-cylinder arrangement allows the cylinders to be made small, giving highly precise ink flow, while the reservoir remains large enough for practical manufacturing use.

In some embodiments, the system described herein utilizes an ultrasonic jet to deposit the ink. In some embodiments, a non-contact jet valve delivers faster, precise dispensing over smooth and uneven surfaces with less turbulence for greater fluid deposit consistency, placement, and process control. For example, the system can dispense micro-deposits as small as 0.5 nL at up to 1000 Hz continuous with up to 1500 Hz maximum bursts. Variable stroke provides better stroke control, making it possible to set very exact, repeatable deposit quantities. Improved close time, in addition to faster full stroke open time, make the system one of the most robust jet dispensing valves on the market. Its modular design and exchangeable parts make it possible to dispense a wider variety of fluids with low to high viscosities. The device can also have the capability to jet low-to high-viscosity fluids provides the flexibility to meet changing dispensing needs.

A variety of designs can also be used for the ink tip. In some embodiments, a standard conical pen tip can be used. Conical pen tips can be easily fabricated from plastics, metals, or ceramics depending on the application requirements for wear resistance and cost. The bore, or orifice, of the pen tip is used to deposit the ink, and it can be straight, linearly tapered, or varied in diameter in a more complex fashion. Regardless of the details of the bore, a characteristic of the conical design is its symmetry. The width of the printed feature will be independent of the direction of travel of the pen tip through space.

In applications where the types of features to be printed are specified, fixed, and relatively uniform in characteristic dimensions, it may be possible to improve manufacturing productivity through the selection of alternative pen tip shapes. For example, if a printed layer is dominated by traces of a specific width, it may be advantageous to employ the rectangular pen tip. This allows all features of the dominant width, for example, corresponding to the long dimension of the pen tip, to be printed rapidly in a single pass. However, there are limitations with this approach. The instantaneous line width and thickness of the printed trace becomes a function of the angle between the direction of motion and the axes defined by the long and short sides of the pen tip. Features that do not have the dominant line width or are angled with respect to the pen tip axes may require very complex tool paths. In the printing of circular or annular features, for example, the line width and thickness change continuously.

After the ink application process, the balloon is placed in an oven to evaporate and eliminate the solvent and solidify the ink. For example, after inking, the modular racks can be placed in a low temperature drying oven (e.g., approximately 120-140 degrees F.) where the balloons are dried. Specifically, the ink is dried, and the residual solvents within the ink are released from the implant.

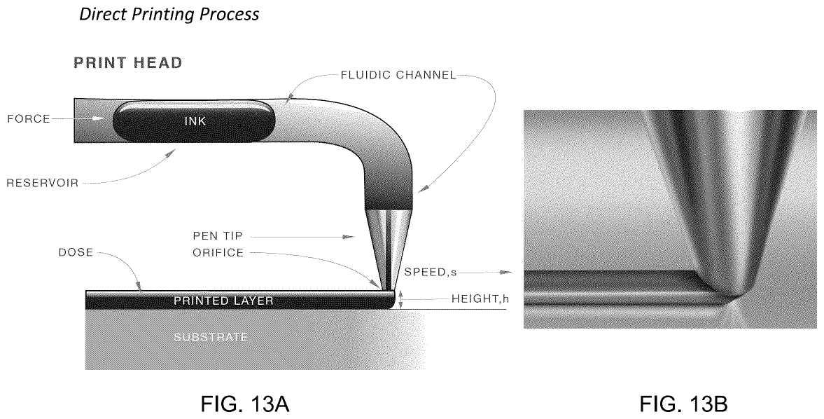

A basic exemplary schematic of the hardware required for direct capillary printing is illustrated in FIGS. 13A and 13B. A quantity of the ink to be printed is held in a reservoir. Inks can vary in viscosity from watery to tar-like. However, the ink must be capable of flowing under the influence of an applied force. The applied force causes the ink to be transported from the reservoir through a fluidic channel to a pen tip that includes a fine opening, or orifice, where it is extruded onto a substrate. The ink reservoir, fluidic channel, pen tip, and the hardware required to create the applied force are generally mounted together and form the print head. A motion control system generates relative movement between the print head and the substrate. The position of the pen tip through space can be described by the instantaneous relative speed of the print head, s, and the instantaneous height of the orifice above the substrate, h. The printing process leaves a “dose” of ink on the substrate that can be measured in mass per unit length of travel of the print head. The dose directly correlates with the instantaneous volumetric flow rate of ink. Not shown are the CAD and CAM systems used to digitally define the tool path that the motion system will execute in order to render the desired printed features.

The XYZ positioning of the piezo-electric head of the inking head assembly ensures the correct position of the nozzle relative to the surface of each balloon. For example, a balloon can have a compound shape such that the dimensions of the balloon can change along its length, and the inking head assembly is configured to account for these changes in balloon shape and size. For example, a balloon can be fatter at one end and thinner at the other end, and the system can move along the length of the balloon and maintain a consistent distance/height from the balloon as the size and/or shape of the balloon changes. In some embodiments, the inking head includes one or more sensors (for example, an optical camera) for optical orientation that are configured to register the location of the rack and the balloons thereon. This also allows for correct orientation of the tip of the inking head in relative to the surface of the balloon (apex). The optical camera can be positioned on the inking head in various locations. For example, the optical camera can be offset from the inking head to prevent the camera from interfering with deposition of the ink on the balloons.

In some embodiments, the system utilizes a software package that is programmed to control the robotic arm/inking head assembly. For example, the software can be used to establish a home position for the inking head.

In some embodiments, the software can control positioning in both horizontal (X) and vertical (Y) positions from a home position. In some embodiments, the software can control positions from the X, Y, and Z directions from a home position. The camera or other sensor information can be used to run refinement of the position of the inking head, for example, by holding the head on the apex of the curve of the balloon, as well as maintaining a specific height from the balloon.

In some embodiments, inking can occur at the apex of the curve of the ballons so that the X and the Y provide the orientation of the ink jet. For example, the X orientation can be running the length of the balloon, and the Y orientation can be running across the balloon. Thus, with a curved balloon, the tip of the inker head, and the location of the ink application, is in a position on the tangent of the balloon (specifically the Y axis) as the ink will be best positioned without any distortion. When the ink is applied 90 degrees to the direction of the ink, falling of the midline, distortion can be induced. Thus, the X axis should be running right down the midline of the balloon and the Y axis should be similarly in the middle of the balloon, applying ink to the tangential surface of the balloon.

In some embodiment, height can affect the spray pattern. The height can be held static during the application, otherwise the pattern of the ink can be affected (for example, this relates to the inverse square law/Hooks Law, relating a decrease in intensity due to distance). Regarding the Z position and the ability for the system to maintain position, this can be an issue with balloon shapes that are not tubular and/or ballons having a diameter that changes over length thereof. For example, a 22/13 mm balloon can be used that holds a 22 mm diameter for a defined length, and then taper down to a 13 mm diameter. Due to the control of the location of the inker head relative to the balloon surface, the system has the ability to hold position over these dimensional changes. Thus, the system can accommodate for balloons with any shape and size and with any dimensional changes along the length of the balloon.

In some embodiments, the system can include a user interface configured to receive inputs relating to operating parameters of the inking head and the robotic arm. This can include movement of the robotic arm/inking head, and inking parameters such as fluid type, viscosity information, and dispensing characteristics of the ink.

For example, a computer system can be programed to control various components of the system, including, for example, control over the inking head, control over the robotic arm, execution of application specific software, and similar operations. In some embodiments, a computer system may be programmed to perform the steps of the methods of the present disclosure and control various parts of the instant systems to perform necessary operation to achieve the methods of the present disclosure. In some embodiments, the processor may be programmed to control the inking head and/or the robotic arm. The X/Y/Z orientation of the inking head assembly can be used to resolve the issue of tip position, with continual adjustment to ensure line conformity.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application.

For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, and/or objects. For example, such projections may be a visual overlay for a mobile augmented reality (MAR) application.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

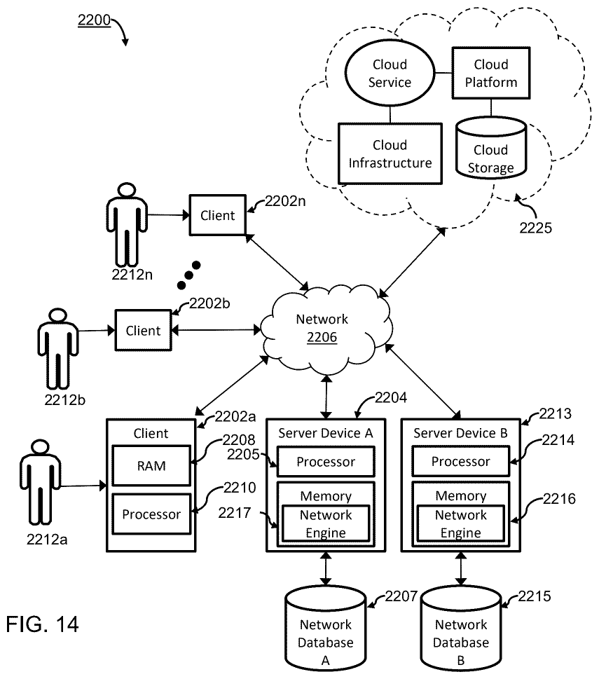

FIG. 14 depicts a block diagram of an exemplary computer-based system and platform 2200 associated with the robotic arm/inking head in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, client devices 2202a, 2202b through 2202n each at least include a computer-readable medium, such as a random-access memory (RAM) 2208 coupled to a processor 2210 or FLASH memory. In some embodiments, the processor 2210 may execute computer-executable program instructions stored in a memory 2208. In some embodiments, the processor 2210 may include a microprocessor, an ASIC, and/or a state machine. In some embodiments, the processor 2210 may include, or may be in communication with, media, for example computer-readable media, which stores instructions that, when executed by the processor 2210, may cause the processor 2210 to perform one or more steps described herein. In some embodiments, examples of computer-readable media may include, but are not limited to, an electronic, optical, magnetic, or other storage or transmission device capable of providing a processor, such as the processor 2210 of client device 2202a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, client devices 2202a through 2202n may also comprise a number of external or internal devices such as a mouse, a CD-ROM, DVD, a physical or virtual keyboard, a display, or other input or output devices. In some embodiments, examples of client devices 2202a through 2202n (e.g., clients) may be any type of processor-based platforms that are connected to a network 2206 such as, without limitation, personal computers, digital assistants, personal digital assistants, smart phones, pagers, digital tablets, laptop computers, Internet appliances, and other processor-based devices. In some embodiments, client devices 2202a through 2202n may be specifically programmed with one or more application programs in accordance with one or more principles/methodologies detailed herein. In some embodiments, client devices 2202a through 2202n may operate on any operating system capable of supporting a browser or browser-enabled application, such as Microsoft™, Windows™, and/or Linux. In some embodiments, client devices 2202a through 2202n may include, for example, personal computers executing a browser application program such as Microsoft Corporation's Internet Explorer™, Apple Computer, Inc.'s Safari™, Mozilla Firefox, and/or Opera.

In some embodiments, through the member computing client devices 2202a through 2202n, users, 2212a through 2212n, may communicate over the exemplary network 2206 with each other and/or with other systems and/or devices coupled to the network 2206. As shown in FIG. 14, exemplary server devices 2204 and 2213 may include a processor 2205 and a processor 2214, respectively, as well as a memory 2217 and a memory 2216, respectively. In some embodiments, the server devices 2204 and 2213 may be also coupled to the network 2206. In some embodiments, one or more client devices 2202a through 2202n may be mobile clients.

In some embodiments, at least one database of exemplary databases 2207 and 2215 may be any type of database, including a database managed by a database management system (DBMS). In some embodiments, an exemplary DBMS-managed database may be specifically programmed as an engine that controls organization, storage, management, and/or retrieval of data in the respective database.

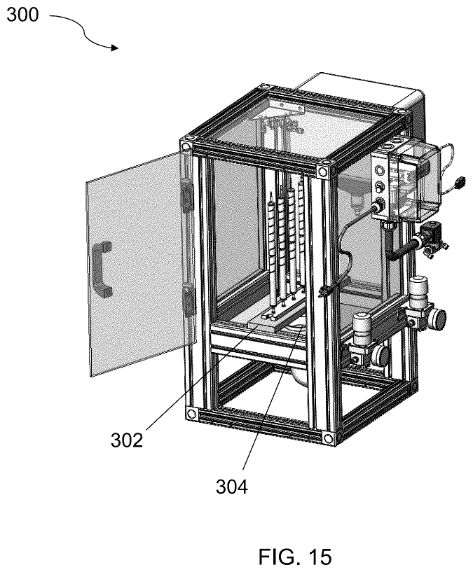

The system can also include optional steps to prepare the balloons for inking. In some embodiments, the system can include a mechanism to clean the balloons before inking. As shown in FIG. 15, the system can include a cleaning tank 300 (i.e., a spritzer tank), or alcohol station, in the form of a contained enclosure that rotates the racked balloons while atomized ethanol or other cleaning agent is applied to the balloons for the purpose of cleaning them before the inking process. The alcohol station can remove any finger grease, oils, or other contaminants after the balloon are loaded onto the modular rack. In some embodiments, the cleaning tank 300 can include a platform 302 for holding one or more rod assemblies thereon. For example, the rod assemblies can be positioned on the platform such that each rod assembly is oriented vertically. The platform holds the rod assembly and rotates them for uniform cleaning.

The amount of ethanol delivered to each balloon and the around of time of the delivery can be controlled based on various factors, including size of the balloon. The balloons are rotated in the tank to uniformly apply the cleaning solution. After the ethanol delivery, the tank is evacuated, through a suction port 304 on the bottom of the tank. In some embodiments, the residual ethanol can be captured in a return tank.

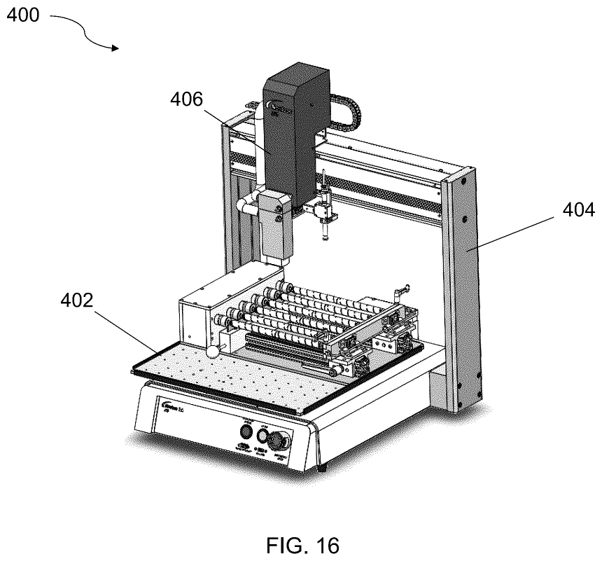

In some embodiments, the system can corona-treat the balloon with a corona discharge treatment device 400 after cleaning, as shown in FIG. 16. A corona discharge treatment can be used to improve the wettability, adhesion, and biocompatibility of a polymeric surface, without affecting the bulk properties. In some embodiments, the corona discharge treatment device can include a base 402 configured to receive the modular rack assembly and a frame 404 coupled to the base such that a corona treatment element 406 can be positioned over the modular rack assembly to apply treatment thereto. The corona treatment 406 is configured to be slidable along the upper portion of the frame to move over the modular rack assembly.

Corona treatment is a surface modification technique that uses a low temperature corona discharge plasma to impart changes in the properties of a surface. The corona plasma is generated by the application of high voltage to an electrode that has a sharp tip. The plasma forms at the tip. A linear array of electrodes is often used to create a curtain of corona plasma. Materials may be passed through the corona plasma curtain in order to change the surface energy of the material. All materials have an inherent surface energy. Thus, a corona discharge is a process by which a current flows from an electrode with a high potential into a neutral fluid, usually air, by ionizing that fluid so as to create a region of plasma around the electrode. The ions generated eventually pass the charge to nearby areas of lower potential or recombine to form neutral gas molecules.

To be more precise, corona treatment is used to increase a material's surface tension in an effective way. Post treatment, the surface can become more impressible to adhesives, inks and coatings.

When the potential gradient (electric field) is large enough at a point in the fluid, the fluid at that point ionizes and it becomes conductive. If a charged object has a sharp point, the electric field strength around that point will be much higher than elsewhere. Air near the electrode can become ionized (partially conductive), while regions more distant do not. When the air near the point becomes conductive, it has the effect of increasing the apparent size of the conductor. Since the new conductive region is less sharp, the ionization may not extend past this local region. Outside this region of ionization and conductivity, the charged particles slowly find their way to an oppositely charged object and are neutralized.

Corona discharge forms only when the electric field (potential gradient) at the surface of the conductor exceeds a critical value, the dielectric strength or disruptive potential gradient of the fluid. In air at sea level pressure of 101 kPa, the critical value is roughly 30 kV/cm.

Coronas may be positive or negative. This is determined by the polarity of the voltage on the highly curved electrode. If the curved electrode is positive with respect to the flat electrode, it has a positive corona; if it is negative, it has a negative corona. A reason for considering coronas is the production of ozone around conductors undergoing corona processes in air. A negative corona generates much more ozone than the corresponding positive corona.

In some embodiments, the corona treatment system can include a hood positioned around the corona treatment system. In some embodiments, the system can also include a sensor or other measurement device that is configured to measure ozone. The system can also include a venting mechanism configured to vent out the ozone that is created during the corona treatment process.

The lifetime of a corona treatment depends on the treated material and the storage conditions. Since the effects of a corona treatment tend to degrade over time, the treatment is usually done shortly before the printing, coating, or bonding process begins. This “in line production” method can allow for the corona treatment to be applied immediately prior to the inking process, such that the surface is primed and ready for the ink.

The material, such as the surface of the balloons, can be positioned in front of an electronic corona discharge. This results in the breakage of oxygen molecules into an atomic form. The atoms are thus allowed to bond with the molecule ends present in the material that's being treated. Hence, the surface of that material becomes chemically active.

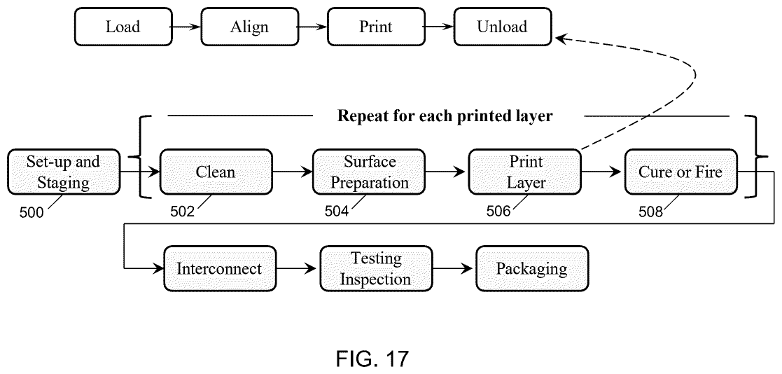

A method for depositing ink on a substrate, such as a balloon, is shown in FIG. 17. The first step in the sequence is the setup of equipment (in step 500), includes loading inks into print heads, programming the required tool paths into the computer controllers, and placing the substrates onto mandrels or holding fixtures as an aid to handling, transporting, and aligning the parts. Each printed layer involves some or all of the four major steps outlined in brackets in the figure. Products having N distinct layers will require N repetitions of the printing steps. Cleaning procedures (in step 502) are substrate dependent and can range from solvent cleaning of polymeric substrates, to ultrasonically activated aqueous cleaning of metals, to high temperature firing of ceramics. Surface preparation techniques (in step 504) include adhesion promotion layers and treatment with liquid chemical agents and plasmas. After the printing step (in step 506), the ink is usually stabilized by curing or firing (in step 508).

### DEVICE EXAMPLES

An extensive range of medical devices have been researched and commercialized using DCP including ablation probes for endoscopies, instrumented endotracheal tubes for neuromonitoring or vital sign measurement, and radiopaque (RO) markings for device visualization under fluoroscopy.

Various types of medical devices can be fabricated using DCP. As mentioned above, the inking process described herein can be used with balloon-based devices that includes radiopaque markings for a bone fracture repair to align and/or immobilize fractured bone.

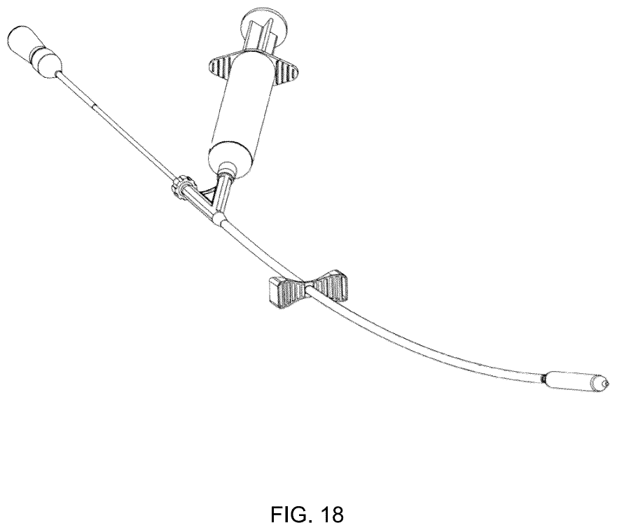

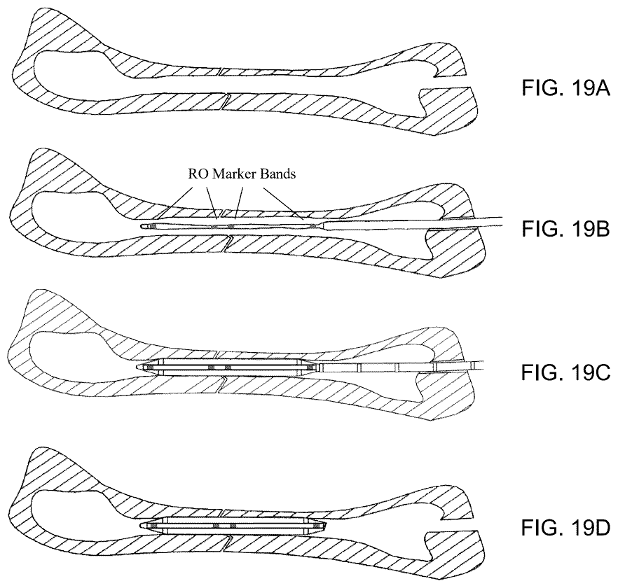

For example, as shown in FIG. 18 and FIGS. 19A-19D, a balloon is affixed to the distal end of a specialized catheter used for bone repair. The catheter contains an optical light guide and a fluidic passageway. The optical light guide delivers short wavelength visible light from an external optical source and distributes it laterally and uniformly over the length of the balloon. The fluidic passageway allows the balloon to be filled with a photopolymerizable resin from a syringe fitted to a port on the catheter located outside of the patient. The catheter is inserted into the intramedullary canal with the balloon in a deflated state (FIG. 19B). After properly positioning the balloon relative to the fracture, the balloon is filled with the resin, which is then optically cured (FIG. 19C). The final steps are to sever the catheter, leaving the implant behind (FIG. 19D), and close up the incision. The visualization features inked onto the surface of the balloon as described herein can be used during this procedure to aide in placement and positioned of the implant relative to the bone.

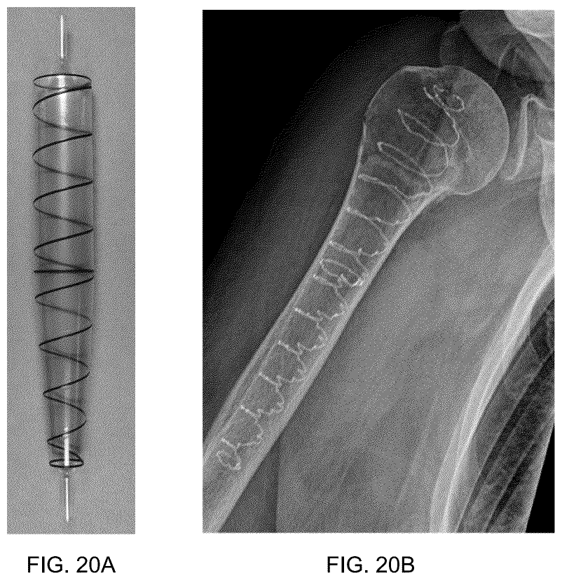

DCP can be used to print a radiopaque marker, in the form of a helix, directly onto the balloon itself. This approach gave excellent visualization of the extent, position, and unfurling of the balloon and eliminated the cost and labor of RO bands. The helical marker was printed using a tungsten-filled ink. FIG. 20A shows an exemplary embodiment of a completed balloon after the marking process with a radiopaque helical marker band, and FIG. 20B shows an exemplary fluoroscopic image of a successful clinical procedure after implantation of the balloon in a fractured or damaged bone.

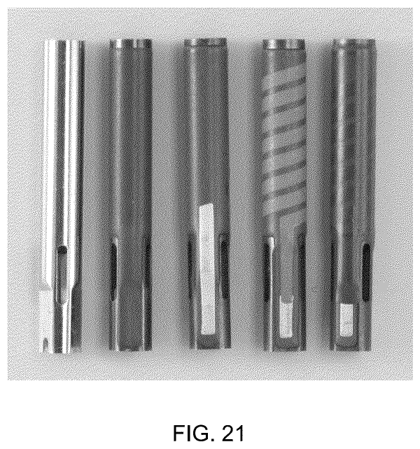

DCP can be used for building customized, high-performance heaters of all descriptions. A medical heater suitable for ablation procedures is shown in FIG. 21. The device is built on a stainless-steel tube with various machined features designed to simplify assembly into the final product. FIG. 21 illustrates exemplary steps in the printing process. First, a dielectric layer is applied to the tube to isolate the energized components. Next, the electrical contact pads and the helical resistive heater trace are applied. Finally, the heater is encapsulated with a hermetic sealant.

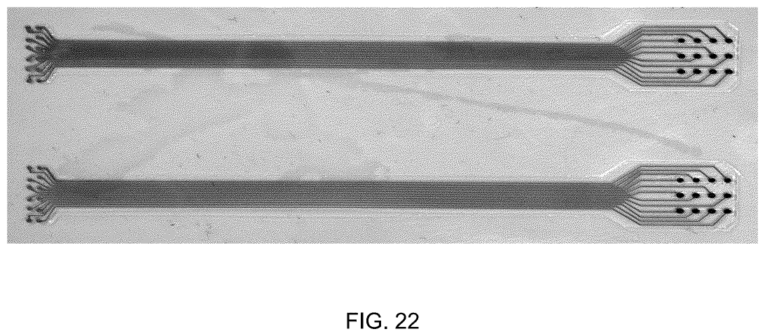

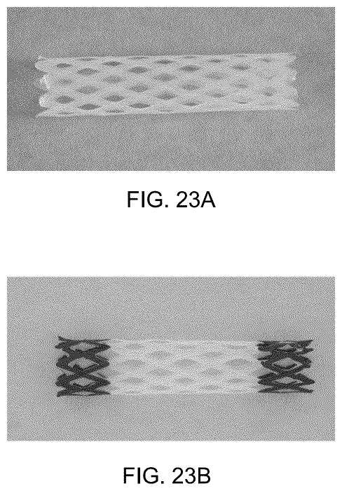

DCP can be used to create entire devices, substrates included. For example, a flexible electrode array suitable for neurosensing and neurostimulation is shown in FIG. 22. The silicone substrate was printed on a flat, hard surface that the ink would wet but not bond to permanently. After curing the substrate, an array of silver-based conductive electrodes and connecting wires were printed. A second silicone overcoat was deposited to insulate the connecting wires. After final curing, the electrode array was peeled off of the working surface. For example, exemplary printed bioabsorbable polymeric stents are shown in FIGS. 23A and 23B. Construction proceeded in a manner similar to that of the flat electrode array. Successive layers of material were printed onto a cylindrical mandrel. FIGS. 23A and 23B shows both structural as well as radiopaque regions of the device (seen in FIG. 23B).

Dispensing of ink using the devices and processes described herein allows for precise, repeatable deposits of ink independent of part topography or tolerance. For example, ink can be dispensed at a continuous speed of up to 1,000 deposits per second. The ink can be deposited from any direction including horizontal and upside down and can meet exact deposit tolerances as small as +/−1%. The system described herein eliminates substrate damage since there is no contact with the surface of the material of the balloons while dispensing the ink. Laser-based light barriers can count every deposit jetted, adding a level of process verification and quality control not possible with contact dispensing. The system also eliminates dispense tip damage, since there is no contact with the surface.

While the presently disclosed embodiments have been described with reference to certain embodiments thereof, it should be understood by those skilled in the art that various changes may be made and equivalents may be substituted without departing from the true spirit and scope of the presently disclosed embodiments. In addition, many modifications may be made to adapt to a particular situation, indication, material and composition of matter, process step or steps, without departing from the spirit and scope of the present presently disclosed embodiments.

We claim:

1. A device, comprising:
a modular rack system configured to receive one or more expandable members, the modular rack system comprising one or more elongate rods assemblies, each rod assembly being sized and shaped to receive a single expandable member; and
an inking system configured to apply one or more visualization features on the one or more expandable members,
wherein the inking system is configured to apply the one or more visualization features to each of the one or more expandable members such that each of the one or more expandable members has identical visualization features thereon.

2. The device of claim 1, wherein the one or more visualization features on each of the one or more expandable members has substantially equal line thickness and width along a length of each of the one or more expandable member.

3. The device of claim 1, wherein the inking system further comprises an inking head assembly configured to move over the modular rack system for applying the one or more visualization features to the one or more expandable members.

4. The device of claim 3, further comprising a robotic arm configured to slidably move along a length of a frame positioned over the modular rack assembly to pass over the expandable members to apply the one or more visualization features.

5. The device of claim 1, wherein the one or more elongate rod assemblies comprises:
a rod configured to receive and support one of the one or more expandable members;
a first end cap configured to couple the rod to a first end of the modular rack system; and
a second end cap configured to couple the rod to a second end of the modular rack system.

6. The device of claim 1, further comprising an air manifold positioned on a first end of the modular rack assembly, the air manifold configured to couple to each rod assembly to provide air for expansion of the one or more expandable members.

7. The device of claim 6, wherein the air manifold is configured to provide air during application of the one or more visualization features.

8. The device of claim 1, further comprising a drive mechanism coupled to each rod of the rod assemblies on which each of the one or more expandable members is positioned that is configured to rotate each of the one or more expandable members.

9. The device of claim 8, wherein the drive mechanism is configured to rotate each of the one or more expandable members at an identical speed.

10. The device of claim 8, wherein the drive mechanism comprises a series of gears configured to rotate the one or more rod assemblies.

11. The device of claim 8, wherein the drive mechanism comprises a series of gears and a belt, the belt being configured to rotate the series of gears to rotate the one or more rod assemblies.

12. A device, comprising:
a modular rack system configured to receive one or more expandable members, the modular rack system comprising one or more elongate rods assemblies, each rod assembly being sized and shaped to receive a single expandable member;
an inking system configured to applying one or more visualization features on the one or more expandable members; and
a drive mechanism coupled to each of the elongate rod assemblies, the drive mechanism being configured to rotate each elongate rod assembly uniformly such that each expandable member is rotated at an identical speed,
wherein the inking system is configured to apply the one or more visualization features to each of the one or more expandable members such that each of the one or more expandable members has identical visualization features thereon.

13. The device of claim 12, wherein the drive mechanism comprises a series of gears configured to rotate the one or more rod assemblies.

14. The device of claim 12, wherein the drive mechanism comprises a series of gears and a belt, the belt being configured to rotate the series of gears to rotate the one or more rod assemblies.

15. The device of claim 12, wherein the inking system further comprises an inking head assembly configured to move over the modular rack system for applying the one or more visualization features to the one or more expandable members.

16. A method, comprising:
prepare one or more expandable members for receiving one or more visualization features by applying the one or more expandable members to one or more rod assemblies;
positioning the one or more rod assemblies on a modular rack;
rotating the one or more expandable members using a drive mechanism coupled to the rod assemblies and the modular rack; and
applying one or more visualization features to the one or more expandable members, the one or more visualization features being applied to each of the one or more expandable members such that each of the one more expandable members has identical visualization features thereon.

17. The method of claim 16, further comprising inflating each of the one or more expandable members using an air manifold coupled to the rod assemblies.

18. The method of claim 16, wherein the one or more visualization features are applied during inflation of the one or more expandable members.
